(19) United States
(12) Patent Application Publication
(10) Pub. No.: US 2016/0353994 A9
(43) Pub. Date: Dec. 8, 2016

(54) Method for the analysis of progression of heterotopic ossification by Raman spectroscopy

(71) Applicants: Nicole Crane, Washington, DC (US); Eric A. Elster, Kensington, MD (US); Jonathan Forsberg, Kensington, MD (US); Douglas Tadaki, Frederick, MD (US)

(72) Inventors: Nicole Crane, Washington, DC (US); Eric A. Elster, Kensington, MD (US); Jonathan Forsberg, Kensington, MD (US); Douglas Tadaki, Frederick, MD (US)

(21) Appl. No.: 14/146,334

(22) Filed: Jan. 2, 2014

Related U.S. Application Data
(60) Provisional application No. 61/748,900, filed on Jan. 4, 2013.

(51) Int. Cl. A61B 5/00; A61B 10/02; A61B 6/00; G01N 21/65; G01N 33/483

(52) U.S. Cl. CPC A61B 5/0075; G01N 21/65; G01N 33/4833; A61B 5/0084; A61B 5/4504; A61B 5/4842; A61B 6/505; A61B 6/5217; A61B 10/02; A61B 5/4519; G01J 3/4412

(57) ABSTRACT

A method for detecting and monitoring the progression of heterotopic ossification by Raman spectral analysis. Analysis of heterotopic ossification progress can be conducted using invasive or invasive means using specific Raman spectroscopy. Analysis is by determination of a number of Raman spectral parameters including the area under one or more of the vibrational bands, band area ratios, band height, ratios of band heights and shift in band center.

### CROSS-REFERENCES TO RELATED APPLICATIONS

This application claims the benefit of U.S. Provisional Application, No. 61/748,900, filed Jan. 4, 2013.

### FIELD OF THE INVENTION

The inventive subject matter relates to a method of determining the progression of heterotopic ossification using Raman spectroscopy.

### BACKGROUND OF INVENTION

Rapid Heterotopic ossification (HO) is defined as the formation of mature lamellar bone in soft tissues. HO has emerged as an important barrier to functional mobility and return to full function and health following a trauma, such as surgery.

Unfortunately, conventional means of primary prophylaxis, external beam radiotherapy, and non-steroidal anti-inflammatory drugs (NSAIDS) produce side effects (mainly related to wound- and fracture healing) that are prohibitively undesirable in this patient population (Coventry and Scanlon, J. Bone and Joint Surg., 63-A: 201-208 (1981); Ritter and Sieber, clin Orthop., 196: 217-225 (1985). As such, they cannot be recommended for widespread use in all patients sustaining high-energy penetrating extremity trauma, illustrating the need for accurate means of risk-stratification.

Many wounds require a series of debridement procedures. These are generally continued three times per week until definitive wound closure using local tissue, flaps or skin grafts can be attempted. Regions where tissues will develop HO frequently become evident during the first 3 to 4 weeks (Crane, et al., Bone 57: 335-342 (2013)).

Experienced surgeons have learned to recognize changes in the physical properties of tissue that are indicative of HO, including a thickening and stiffening of tissues, evident during debridement procedures. Early mineralization of these tissues can be felt when debriding with a surgical knife, however, once mineralization occurs (usually weeks after injury), conventional means of primary prophylaxis are typically ineffective. Furthermore, efforts to preserve residual limb length and/or to provide durable muscular coverage over fractures preclude excision of all tissues exhibiting early ectopic bone formation, and thus, development continues until the bone is mature. If the HO becomes symptomatic, surgical excision remains the only option, which carries unique preoperative complications. Nevertheless, if wound-specific changes could be identified earlier, prophylactic measures could be brought to bear at a time thought to influence osteoblastic differentiation.

### SUMMARY OF INVENTION

An object of the invention is a method to determine the presence, maturity or composition of heterotopic ossification using one or more Raman spectral measurements.

The contemplated measurements in the method include one or more of the following: band area ratios, band height, ratio of band height, increase or decrease of band center or band area ratio. The determination of heterotopic ossification (HO) is generally made by comparing Raman spectral measurements compared to normal soft tissue or normal bone.

In one embodiment the progression of HO using Raman spectroscopy. The method comprises measuring and monitoring specific Raman band ratios, such as 1445 cm⁻¹, 945 cm⁻¹; 960 cm⁻¹; and 1070 cm⁻¹; and wherein two or more of said band areas are used in computing band area ratios, wherein said band area ratios are selected from the group consisting of 945/960 cm⁻¹, 1070/960 cm⁻¹, 960/1445 cm⁻¹, and 1070/1445 cm⁻¹. In another embodiment, the method contemplates the determination of HO and its compositional maturity by measuring one or more band area ratios selected from the group consisting of 1660/1445 cm⁻¹, 1680/1445 cm⁻¹, 1640/1445 cm⁻¹, 1640/1660 cm⁻¹, 1240/1270 cm⁻¹, and 1340/1270 cm⁻¹, and the difference in these parameters compared to normal soft tissue, such as muscle, or bone.

It is contemplated that the determinations in the inventive method can be conducted by invasive or noninvasive techniques. Invasive techniques include, for example, obtaining tissue biopsy samples for use in conducting Raman analysis. Noninvasive techniques include, for example, the employment of fiber optic probes.

### BRIEF DESCRIPTION OF DRAWINGS

FIG. 1. Bone progression associated with 960 cm⁻¹ band width (at half maximum).

FIG. 2. Raman spectroscopy of tissue biopsies. Panel A shows the Raman spectra of normal muscle and type I collagen, type II collagen, and type IV collagen. Panel B shows the Raman spectra of normal muscle verses unmineralized and mineralized HO. Shaded areas indicate regions where vibrational bands are significantly different.

FIG. 3. Comparison of Raman vibrational band area ratios for matrix components of tissue.

FIG. 4. Comparison of Raman vibrational band area ratios for mineral components of tissue. In FIG. 4, the statistically significant differences (p<0.0125) are indicated by an asterisk.

FIG. 5. Use of mineral band area ratios for monitoring progression of HO. Comparison of Raman vibrational band area ratios for mineral components from HO collected from 60 days post-injury (or earlier) (Cases 1 and 2) up to approximately 6 months to one year post-injury (cases 3-5).

### DETAILED DESCRIPTION OF PREFERRED EMBODIMENTS

Current methods for monitoring wound healing rely largely on clinical observations. These observations include evaluation of wounds by evaluating evaluated parameters such as location of injury, adequacy of perfusion, gross appearance of the wound, wound tensile strength, and the patient's general condition. Since parameters, such as adequacy of perfusion and tensile strength are not readily quantifiable during surgery, evaluation of wound healing can entail a significant amount of subjectivity, which, when applied by inexperienced physicians, can lead to errors in assessment. As such, a need for non-invasively and more objectively assessing healing is needed.

Raman spectroscopy is a non-invasive scattering technique that can be used to obtain information about the structure and composition of molecules from their vibrational transitions. Therefore, through determination of a Raman spectrum of a tissue, a chemical “fingerprint” of the tissue can be obtained. Table 1 illustrates Raman spectral bands and assignments for collagen, muscle and bone.

TABLE 1¹

| ν (cm⁻¹) | Band Assignment | Component |
|---|---|---|
| 593 | ν₄ PO₄³⁻ bending | hydroxyapatite |
| 821 | ν(CC) of backbone | collagen; muscle |
| 856 | ν(CC) of hydroxyproline ring | collagen; muscle |
| 873 | ν₃ P—OH stretching | bone |
| 876 | ν(CC) of hydroxyproline ring | collagen; protein |
| 921 | ν(CC) of proline ring | collagen; protein |
| 938 | ν(CC) of protein backbone | collagen; muscle; protein |
| 945-952 | ν₁ PO₄³⁻ stretch | amorphous calcium |
| | | phosphate |
| 959 | ν₁ PO₄³⁻ stretch | hydroxyapatite |
| 1004 | ν(CC) aromatic ring | Phe; collagen; muscle |
| 1032 | ν₃ PO₄³⁻; ν(CC) skeletal; | bone; collagen; muscle |
| | C—O stretch | |
| 1071 | ν₁(CO₃²⁻) | bone |
| 1075 | ν₃ PO₄³⁻ stretch | hydroxyapatite |
| 1080 | ν(CC) and ν(CN) skeletal | collagen; muscle |
| 1159 | ν(CC) and ν(CN) skeletal | carotenoid |
| 1178 | ν(CC) and ν(CN) skeletal | collagen; muscle |
| 1244 | δ(CH₂) wagging; ν(CN) amide | collagen; muscle |
| | III disordered/β-sheet | |
| 1274 | ν(CN) and δ(NH) amide III | collagen; muscle |
| | α-helix | |
| 1297 | δ(CH₂) twisting | collagen; muscle |
| 1343 | γ(CH₂, CH₃) wagging | collagen; muscle |
| 1385 | δ(CH₃) symmetric | collagen |
| 1448 | δ(CH₂) scissoring | collagen; muscle |
| 1524 | carotenoid | collagen; muscle |
| 1552 | ν(CC) ring stretch | collagen; muscle; Trp |
| 1665 | ν(CO) amide I | collagen; muscle |

¹Information is taken from: Wood and Mcnaghton, J. Raman Spectroscopy, 33: 517-523 (2002); Sane, et al., Anal. Biochem., 269: 255-272 (1999); Lippert, et al., J. Am. Chem. Soc., 98: 7075-80 (1976); Maiti, et al., J. Am. Chem. Soc. 126: 2399-2408 (2004); Pezolet, et al., Biophys. J., 53: 319-325 (1988); Wohlrab, et al., Biopolymers, 62: 141-146 (2001); Frushour and Koenig, Biopolymers, 14: 379-391 (1975); Chrit, et al., J. Biomed. Opt., 10: 44007 (2005); and Maquelin, et al., J. Microbiol. Methods, 51: 255-271 (2002)).

### Example 1

### Development of HO and determination of HO composition

Raman spectral parameters can be used to determine the presence or maturity of hetertopic ossification (HO), which is defined as the aberrant formation of mature, lamellar bone in nonosseous tissue. Raman spectroscopy may be valuable in monitoring HO non-invasively (Potter, et al., J. Bone and Joint Surgery, Am., 92 Suppl 2: 74-89 (2010). Use of Raman spectroscopy for monitoring of HO has been suggested (Crane, et al., Proc. SPIE, 7895 (2011)) using band area ratios calculated from matrix bands 1660/1445, 1680/1445, 1640/1445, 1240/1270, and 1340/1270 cm⁻¹. Similarly, band locations, but not band area ratios, has been suggested as a means of monitoring development of HO (Crane and Elster, J. Biomed. Opt. 17(1): 010902 (2012)).

One Raman spectral parameter, band area ratios (BARs), are calculated by dividing the band area of a Raman band (such as for 1070 cm⁻¹) by another band area, for example 1445 cm⁻¹. The association of compositional trends in related tissue/bone samples can be evaluated using the band area ratios. These are illustrated in Table 2.

TABLE 2

| Band Area | |
|---|---|
| Ratio (cm⁻¹⁾ | Compositional trend |
| 960/1445 | Extent of mineralization or mineral-to-matrix (MTMR) |
| 1070/960 | An increase in mineral carbonation |
| 1070/960 and | Mineral carbonation |
| 1070/1445 | |
| 945/960 | Mineral maturity |
| 1240/1270 | Protein order/disorder |
| 1680/1660 | A decrease in reducible collagen crosslinks |
| 1340/1270 | α-helical structure |

As illustrated in Table 3, Raman band centers (for mineral components of HO tissue) at ˜1070 cm⁻¹ and ˜1045 cm⁻¹ can also be used to distinguish early HO from mature HO, in addition to the mineral immaturity and crystallinity band area ratios. Statistical significance is indicated by an asterisk (*=p<0.05).

TABLE 3

| | 1660* | 1445* | 1340.0 | 1270.0 | 1240.0 | 1070* | 1045* | 960.0 | 945.0 |
|---|---|---|---|---|---|---|---|---|---|
| control muscle | 1655.9 | 1445.1 | 1343.0 | 1271.7 | 1244.7 | — | — | — | — |
| injured muscle | 1658.3 | 1445.1 | 1341.9 | 1268.9 | 1246.7 | — | — | — | — |
| early HO | 1658.7 | 1447.6 | 1343.6 | 1271.0 | 1244.4 | 1075.8 | 1059.1 | 960.1 | 944.6 |
| mature HO | 1660.1 | 1449.3 | 1343.8 | 1269.6 | 1244.6 | 1071.9 | 1046.4 | 960.4 | 944.0 |
| normal bone | 1661.3 | 1450.5 | 1343.5 | 1270.1 | 1243.7 | 1072.7 | 1048.7 | 960.3 | 946.6 |

Finally, the heterogeneity of the mineral-to-matrix ratio in bone can be used to distinguish HO bone from normal bone. Measures of MTMR in HO bone have a large standard deviation compared to normal bone MTMRs. Raman spectroscopy can be adapted as an objective, intraoperative, non-invasive means by which to risk stratify wounds, and can be performed in real-time. If Raman spectroscopy demonstrates that a wound has Raman spectral features associated with the formation of HO, prophylaxis could be employed in select cases.

The detection of heterotopic ossification comprises detection of mineralization of tissue using Raman spectroscopy. In this method tissue regions are evaluated for changes in specific Raman vibrational bands. For example, there is a decrease in the 1340 and 1320 cm⁻¹ vibrational bands, compared to uninjured tissue, indicating collagen specific alterations within the tissue due to traumatic injury. Additionally prominence of vibrational bands at 1070, 960, and 591 cm⁻¹ indicate mineralization of the tissue by addition of carbonated apatite. These vibrational bands are associated with phosphate and carbonate stretching modes of bone, indicating early heterotopic ossification of tissue region affected by traumatic injury.

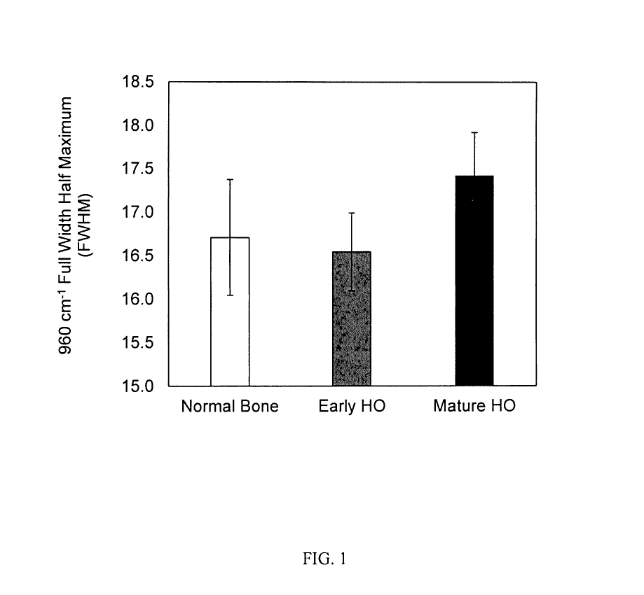

However, detection and monitoring of HO progression, is needed to ascertain maturity of mineralization. Although x-ray evaluation will provide information concerning the presence of HO, it is not capable of providing chemical analysis of the bone formation or chemical changes in bone character. FIG. 1 illustrates the association of the 960 cm⁻¹ band area and progression of HO and normal bone.

In a preferred embodiment, a method for monitoring the progression of HO comprises measuring and monitoring specific Raman band ratios comprising 945/960 cm⁻¹ or the ratio 1070/960 cm⁻¹. The 945/960 cm⁻¹ provides an assessment of mineral maturity. As the 945 cm⁻¹ shoulder decreases, respective to the 960 cm⁻¹ band, bone mineral is maturing and becoming more crystalline or less amorphous. Amorphous mineral is attributable to immature bone. The 1070/960 cm⁻¹ band area ratio provides and assessment of bone maturity. The 1070 cm⁻¹ band is a carbonate band. Bone becomes carbonated as it matures. Therefore, the 1070/960 cm⁻¹ band area ratio increases with bone maturity.

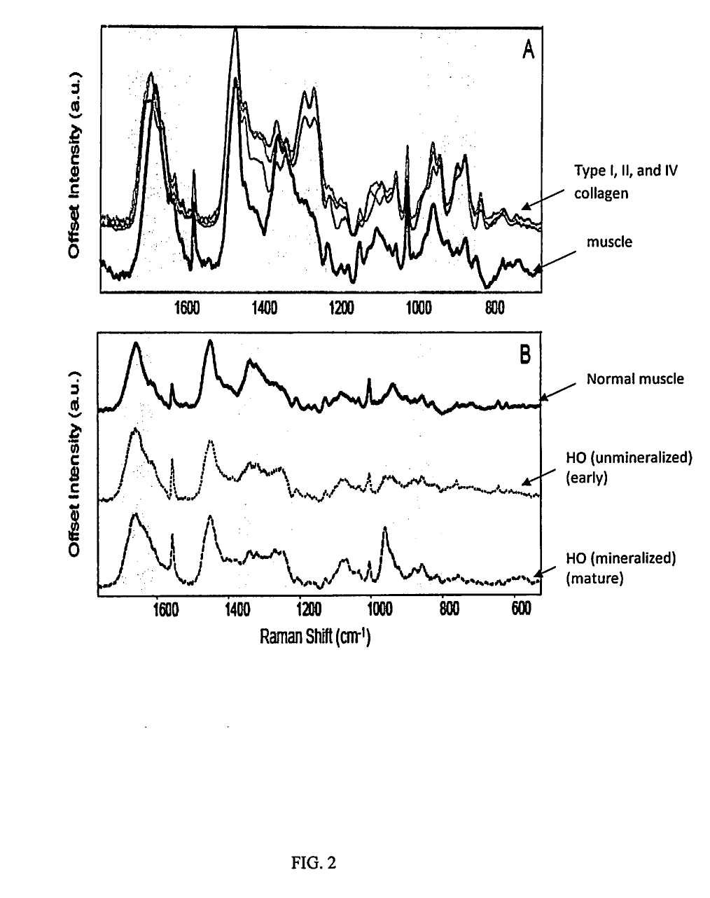

Raman Spectroscopy of various tissues is illustrated in FIG. 2. Types I, II, and IV collagen were examined spectroscopically, along with normal muscle (FIG. 2A). Table 1, above, shows the band assignments. Obvious differences are evident in the 1665 cm⁻¹ band (Amide I), the Amide III envelop (1340, 1320, 1270, and 1245 cm⁻¹), and the 940 cm⁻¹, 920 cm⁻¹, 876 cm⁻¹ and 855 cm⁻¹ bands (C-C backbone stretching). The Raman spectrum of muscle contains an amide I band at approximately 1655 cm⁻¹ while the collagen Amide I band is centered at 1665 cm⁻¹. Additionally, the 1340 and 1320 cm-1 bands are more prominent in the muscle spectrum but the collagen spectra display increased 1270 and 1245 cm⁻¹ spectral bands.

The 876 cm⁻¹ and 855 cm⁻¹ Raman spectral bands of the collagen spectra are more intense than those exhibited in the muscle spectrum. Raman spectra of ex vivo samples of uninjured (or control) muscle, injured muscle, and excised tissue from heterotopic ossification surgical removal were also compared and illustrated in FIG. 2B. FIG. 2B, shows the offset spectra of control muscle sample, a sample of unmineralized HO tissue, and a sample of mineralized HO tissue. The mean band center for the Amide I band of uninjured muscle is 1655 cm⁻¹. For the HO tissue, whether unmineralized or mineralized, the Amide I band shifts to a higher frequency that is centered at 1662-1663 cm⁻¹. Differences are also evident in the Amide III envelope of the spectra. The intensity of the 1340 cm⁻¹ Raman vibrational band is decreased in the spectra of the HO tissue compared to the uninjured muscle tissue. The 1270 cm⁻¹ and 1240 cm⁻¹ Raman vibrational bands are increased in the spectra of the HO tissue compared to the uninjured muscle.

The most notable difference in the spectrum of the mineralized HO tissue is the presence of the 960 cm⁻¹ band, a v₁ P-O stretching mode. This is a typical Raman vibrational band observed for hydroxyapatite, and in this case, for the carbonated hydroxyapatite in bone mineral. Finally, the intensities of the 921 cm⁻¹, 876 cm⁻¹, and 855 cm⁻¹ bands are more intense in the spectra of the HO tissue than in the spectrum of the uninjured muscle.

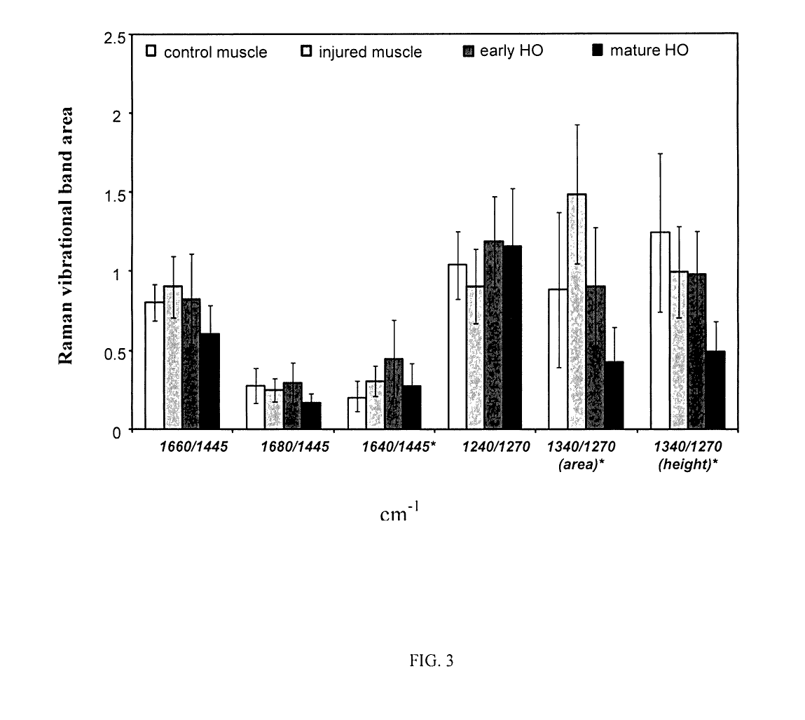

In-depth characterization of the tissue matrix is presented in FIG. 3. Using the 1340/1270 cm⁻¹ band area ratio and the 1640/1445 cm⁻¹ BAR, one can clearly distinguish muscle tissue from HO tissue (early and mature). As bone matures, there is a decrease in the 945/960 cm⁻¹ band area ratio and an increase in the 1070/1445 cm⁻¹ band area ratio. Early HO has the highest mineral immaturity BAR and the lowest mineral carbonation band area ratio.

As illustrated in FIG. 3, calculated band area ratios for the Raman spectra of control muscle (n=3), injured muscle (n=8), unmineralized HO tissue (n=12), and mineralized HO tissue (n=12) for matrix bands. As shown in FIG. 3, there is a statistically significant difference between the 1660/1445 cm⁻¹ band area ratio when comparing uninjured muscle to injured muscle (1.59±0.06 vs. 1.2±0.21, respectively), as well as when comparing muscle tissue and HO tissue (0.90±0.26 vs. 1.01±0.20, respectively). There is also a significant difference between the 1680/1445 cm⁻¹ and 1640/1445 cm⁻¹ band area ratios, when comparing muscle tissue and HO tissue (0.33±0.08 vs. 0.38±0.19, and 0.36±0.09 vs. 0.37±0.08, respectively). Band area ratios for the Amide III envelope also indicate significant differences between the tissue types. The p-values calculated for the 1240/1270 cm⁻¹ band area ratios are <0.01 for the comparison of muscle tissue and unmineralized HO tissue (0.86±0.17 vs. 1.16±0.25, respectively) and <0.02 for the comparison of muscle tissue and mineralized HO tissue (0.86±0.17 vs. 1.29±0.25, respectively).

Notable differences are also demonstrated for the comparison of the 1340/1270 cm⁻¹ band area ratios calculated for muscle tissue and HO tissue (1.64±0.37 vs. 0.91±0.21, respectively—p<0.05), as well as for unmineralized and mineralized HO tissue (1.11±0.19 vs. 0.91±0.21, respectively—p<0.05).

As injured muscle transitions to mineralized tissue, with significant changes in many tissue matrix bands, as illustrated in FIG. 3. First there is a shift in the amide I band in the Raman spectra from 1655 cm⁻¹ to 1660 cm⁻¹. This is likely due to increased collagen content. Second, the band width of amide I band increases (i.e., see the 1660/1445 cm⁻¹ band area ratio in FIG. 3). Bands at 1640 and 1660 cm⁻¹ are assigned to α-helix protein secondary structure while the band at 1680 cm⁻¹ is assigned to β-sheet protein secondary structure. Muscle is composed predominantly of actin and myosin, both of which are more alpha-helical than collagen. As collagen content increases and muscle myofibers degenerate, the band center and width more closely resemble that of collagen (specifically type I). The intensity of amide III bands changes, most notably with an increase in the protein order/disorder band area ratios and a decrease in the α-helical structure band area ratio (see FIG. 3). The Raman band at 1240 cm⁻¹ is attributed to more disordered protein structures, such as those containing a large number of β-pleats or random coils, while the 1270 cm⁻¹ Raman band is assigned to more ordered protein structures, such as those containing a large number of helical coils.

The Raman spectrum of unmineralized HO tissue closely resembles the Raman spectrum of type I collagen. Type I collagen plays an important role not only in wound healing, but also in the formation of osseous tissue, such as HO. Osteoblasts secrete and deposit type 1 collagen, which comprises 90% of bone matrix prior to mineralization. In some cases, the collagen serves as an initiator of wound healing and acts as the scaffold for deposition of bone mineral.

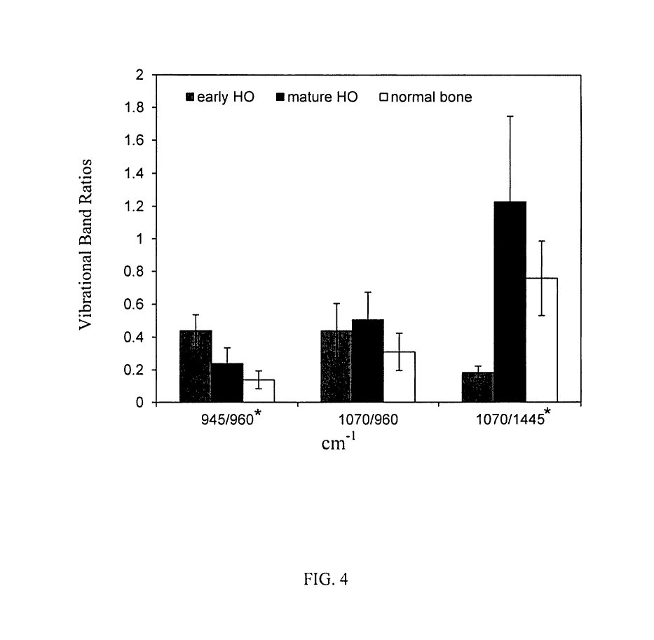

In the inventive method, Raman spectral parameters provide informative insight into the mineralization of soft tissue and HO progression. In one embodiment, the inventive method comprises determining the area under the multiple Raman vibrational bands Raman spectral data at 945 cm⁻¹; 960 cm⁻¹; 1070 cm⁻¹; and 960 cm⁻¹ subsequent to identification of HO regions. The 960 cm⁻¹ phosphate band in the Raman spectrum of bone can be deconvoluted into more than one Raman spectral band, depending on the species of mineral present in the tissue. For example, the presence of a shoulder at 945 cm⁻¹ can be attributed to amorphous calcium phosphate, an uncarbonated mineral. Therefore, an increase in 945 cm⁻¹ shoulder is indicative of immature mineral. As bone matures, this shoulder will decrease. Therefore, the 945/960 cm⁻¹ band area ratio can be used as a measure of mineral maturity, as illustrated in FIG. 4.

As shown in FIG. 4, the least mature HO has the highest 945/960 cm⁻¹ band area ratio. Also, the 1070 cm⁻¹ Raman spectral band is attributed to a v₁ carbonate vibration. As bone matures the incorporation of carbonate into the mineral lattice increases. Increased carbonate content has been associated with increased 1070/1445 cm⁻¹ band area ratio is indicative of increased mineral maturity. Also illustrated in FIG. 4, the 1070/960 cm⁻¹ ratio of early HO and mature HO is 50-100% higher than in normal bone.

### Example 2

### Determination of Progression of HO

When comparing muscle (normal or injured) to HO tissue (early or mature), there is a decrease in the 1660/1445 cm⁻¹ (p<0.03), 1680/1445 cm⁻¹ (p=0.03), and 1340/1270 cm⁻¹ (α-helical structure, p<0.01) band area ratios. This is illustrated in FIG. 3. As shown in FIG. 3, there is also an increase in the 1640/1445 cm⁻¹ band area ratio (p<0.01) and the 1240/1270 cm⁻¹ (protein order/disorder) band area ratio (p<0.10). These changes in BARs can also be examined as a progression of normal tissue to diseased tissue. In the transition from normal to injured muscle, there is: 1) an increase in the 1660/1445 cm⁻¹ and 1640/1445 cm⁻¹ BARs as well as protein order/disorder BARs. In the transition from injured muscle to HO tissue, there is: 1) a decrease in the 1660/1445 cm⁻¹ BAR, the α-helical structure BAR, and an increase in the protein order/disorder BAR.

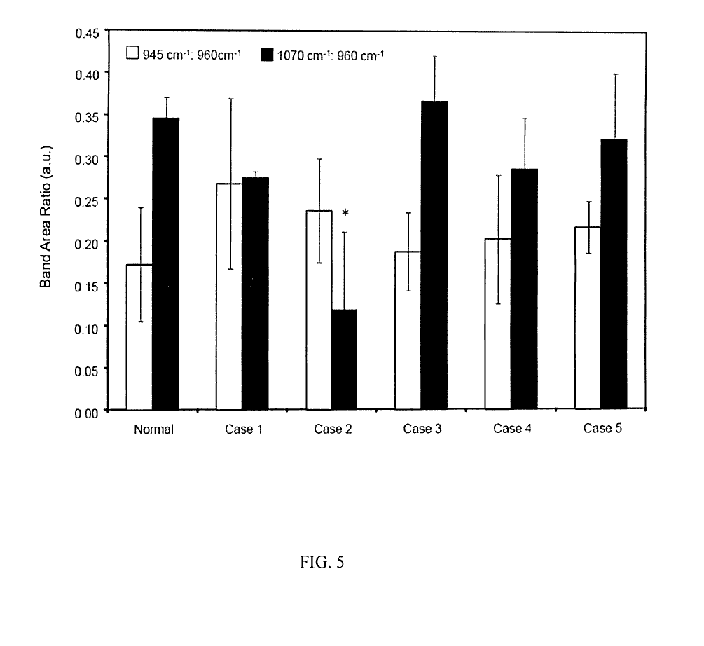

Use of Raman spectroscopy to follow the development of HO progression is illustrated in FIG. 5. For FIG. 5, tissue samples were placed on an aluminum foil covered weighing dish prior to spectral acquisition. A 785 nm Raman PhAT system (Kaiser Optical Systems, Inc., Ann Arbor, Mich.) was used to collect spectra of the tissue biopsies. Final spectra were the accumulation of forty 5 second spectra, acquired using the 3 mm spot size. At least three dark-subtracted, illumination-corrected spectra were obtained for each biopsy/sample. All spectral preprocessing was performed in GRAMS/AI software (Thermo Fisher Scientific, Madison, Wis.). Raman spectra were truncated to 1800-400 cm⁻¹ and baseline corrected with a sixth degree polynomial. Spectral subtraction of blood was performed if spectral interference of blood was noted. All spectra were intensity normalized to the CH₂ scissoring band at 1445 cm⁻¹ Subsequently, curve fitting was performed over three spectral regions, 1730-1500 cm⁻¹, 1525-1185 cm⁻¹, and 1150-900 cm⁻¹. All Raman bands were fit with mixed Gaussian/Lorentzian bands. The fit was considered good when the R² value reached at least 0.99. Differences in band area ratios were assessed using a Mann-Whitney U-test. Analyses were performed using SPSS software (SPSS 18.0, SPSS Inc., Chicago, Ill.). Differences in values were considered statistically significant with a two-tailed p-value less than 0.05.

As mentioned previously and as illustrated in FIG. 4, comparing mineral band area ratios for normal bone (femur) and for HO tissue, the 945/960 cm⁻¹ band area ratio provides a measure of mineral maturity. As illustrated in FIG. 4, as bone matures, the 945/960 cm⁻¹ band area ratio decreases. The 1070/960 cm⁻¹ band area ratio can also be a measure of bone maturity, which increases as the bone matures. The 1070/960 cm⁻¹ ratio has also been correlated with bone mechanical properties such as modulus, yield stress, and fracture stress (Morris, M. D., Clin. Orthop. Relat. Res., 469: 2160-2169). In addition, mineral crystallinity is higher in mature HO samples than in normal bone or early HO samples (p=0.055).

As shown in FIG. 5, the normal bone 945/960 cm⁻¹ and 1070/960 cm⁻¹ band area ratios are 0.17±0.07 and 0.35±0.03, respectively. Band area ratios in normal bone are contrasted in FIG. 5 to cases 1 and 2, which had HO tissue surgically removed soon after post-injury development (less than 60 days) while cases 3-5 underwent HO excision over 100 days post-injury; this is reflected in the Raman spectral band area ratios. Cases 1 and 2 have the highest 945/960 cm⁻¹ band area ratios (mean=0.25±0.02) and the lowest 1070/960 cm⁻¹ band area ratios (mean=0.20±0.1), indicative of a mineral that is not fully matured. This trend is reversed for cases 3-5 where the 945/960 cm⁻¹ band area ratios are lower than cases 1 and 2 (mean=0.20±0.11) and the 1070/960 cm⁻¹ band area ratios are higher than cases 1 and 2 (mean=0.32±0.04). Only case 2 demonstrated a significantly lower 1070/960 cm⁻¹ band area ratio when compared to normal bone (0.12±0.09 vs. 0.35±0.03—p<0.05).

### Example 3

### Method for Monitoring HO Progress

Raman spectroscopy has distinct advantages over other techniques assessing tissue during surgery, such as histology or inspection by the surgeon. Frozen section and/or permanent pathologic analysis can be used to identify early stages associated with HO formation. However, this requires multiple biopsies, is time and labor intensive and may not be sufficiently precise. Also, the ability of the surgeon to identify early HO tissue during a debridement is subjective and relies on personal experience.

In a preferred embodiment, Raman spectral parameters, such as band areas, band area ratios, band height, ratio of band height, and decreases or increases from band center, are compared with other tissue, such as soft tissue (e.g., muscle) or bone in order to determine the composition of the measured tissue and determine the presence and maturity of HO. Shown in Table 4 are Raman spectral parameters associated with the determination of the presence, maturity or composition of HO that can be incorporated into the inventive method.

Measured tissue can be compared to bone or other soft tissue, such as muscle, to compare band area, band area ratio, height/band intensity ratio or band center. Measurements, in the form of ratios of band areas, such as 945 cm⁻¹/960 cm⁻¹ and 1070 cm⁻¹/960 cm⁻¹ are measured. The ratios are then compared to normal bone, or soft tissue, for example muscle. As such, in one embodiment, measurement of 945/960 and 1070/960 cm⁻¹ band area ratios are used to distinguish early HO from mature HO (FIG. 4). A change (increase or decrease) of 945/960 cm⁻¹ ratio can be used distinguish HO from normal bone. Similarly, a ratio 1070/960 cm⁻¹ greater than that for normal bone indicates the presence of HO. An increasing 1070/960 cm⁻¹ ratio is indicative of maturing HO. For example a 1070/960 cm⁻¹ ration that is 40-50% over normal bone indicative of mature HO with increasing incorporation of mineral carbonation. In another embodiment, normal bone is distinguished from mature HO by measurement of the 1070/1445 cm⁻¹ band area ratio, which is also illustrated in FIG. 4 and FIG. 5. In conjunction with the 1070/1445 cm⁻¹ band area ratio, the 960 cm⁻¹ full width half maximum (FWHM) and the 960/1445 cm⁻¹ mineral to matrix ratio. Measurement of the FWHM of the 960 cm-1 v₁ phosphate band can be used to determine mineral crystallinity. The presence of HO and its progression from early to mature HO can be monitored by evaluating various metrics, as illustrated in Table 4.

TABLE 4

| | | (Determination |
|---|---|---|
| Raman-Tissue | Metric (Raman spectra | (presence) of mature |
| comparison | parameters) | or early HO¹) |
| HO compared | 1640/1660 cm⁻¹ band area ratio | HO (early or |
| to muscle | ≧2 times over muscle | mature) |
| tissue | ≧20% decrease in 1340/1270 cm⁻¹ | Early HO |
| | height ratio compared to muscle | |
| | ≧60% decrease in 1340/1270 cm⁻¹ | Mature HO |
| | height ratio compared to muscle | |
| | ≧25% decrease in 1660/1445 cm⁻¹ | Mature HO |
| | band area ratio of muscle or early | |
| | HO | |
| | 2 cm⁻¹ increase (i.e., intensity) | Early and mature |
| | in 1660 cm⁻¹ band center | HO tissue |
| | compared to muscle | |
| | >3 cm⁻¹ increase in 1445 cm⁻¹ | Early and |
| | band center compared to | mature HO |
| | muscle | |
| Early HO | ≧50% decrease in 1340/1270 cm⁻¹ | Mature HO |
| compared to | height ratio and | |
| mature HO | 1340/1270 cm⁻¹ band area | |
| | ratio compared to early HO | |
| | ≧40% decrease in 945/960 cm⁻¹ | Mature HO |
| | band area ratio compared | |
| | to early HO | |
| | ≧40% increase in 945/960 cm⁻¹ | Early HO |
| | band area ratio compared | |
| | to early normal bone | |
| | >3 cm⁻¹ increase from 1070 cm⁻¹ | Mature HO |
| | compared to early HO | |
| | >10 cm⁻¹ increase from 1045 cm⁻¹ | Mature HO |
| | compared to early HO | |
| | >50% decrease in MTMR | Mature HO |
| | (960/1445 cm⁻¹) compared to | |
| | early HO | |
| Mature HO | >60% increase in 1070/1445 cm⁻¹ | Mature HO |
| from normal | band area ratio compared | |
| bone | to normal bone | |
| | >2 cm⁻¹ decrease in 945 cm⁻¹ | Mature HO |
| | band center compared to | |
| | normal bone | |
| | >5% increase 960 cm⁻¹ (full | Mature HO |
| | width half maximum | |
| | (FWHM) (mineral | |
| | crystallinity) compared to | |
| | normal bone | |
| | 50% greater standard | Mature HO |
| | deviation of mineral-to-matrix | |
| | ratio (960/1445 cm⁻¹) | |
| | compared to bone | |
| | ≧40% increase in band area | early to mature HO |
| | ratio of 1070/960 cm⁻¹ | |

¹HO refers to heterotopic ossification

In one embodiment, the results of Raman vibrational band analysis of HO would be collected over a period of time. This data is analyzed to determine the progression of HO and as the wound heals and any treatment for HO proceeds.

The Raman spectral data region includes the area typically to include a surrounding region of a wound, anticipated wound or region receiving a trauma. Determination of traumatized tissue or mapping of the area following detection of ossification can be noninvasive and comprise comparing Raman spectra associated with other tissue verses spectra associated with HO. For example, as discussed above, the intensity of the band at 1340 cm⁻¹ is decreased in the spectra of the HO tissue compared to uninjured muscle tissue.

Therefore, an embodiment of the method comprises, in part, determination of the area under Raman bands 1660 cm⁻¹, 1445 cm⁻¹, 1680 cm⁻¹, 1445 cm⁻¹, 1640 cm⁻¹, 1240 cm⁻¹, 1270 cm⁻¹ and 1340 cm⁻¹. The ratios: 1660/1445 cm⁻¹, 1680/1445 cm⁻¹, 1640/1445 cm⁻¹, 1240/1270 cm⁻¹, and 1340/1270 cm⁻¹ are then determined for monitoring of HO. Additionally, identification of HO areas are identified by measurement and assessment of the area under vibrational bands at 1070, 960, and 591 cm⁻¹ since these are indicative of mineralization of the tissue by addition of carbonated apatite, are added to the assessment, especially early in the mapping of the HO region. It is also possible to calculate band area ratios using the 1003 cm⁻¹ (or 855+875 cm⁻¹) band, instead of the 1445 cm⁻¹ band. Additionally, the intensities of other bands can be included in the evaluation, including at 921 cm⁻¹, 876 cm⁻¹ and 855 cm⁻¹, which are more intense in HO tissue than in uninjured muscle, are evaluated as well, especially in mapping the tissue trauma region. These bands are v_{(CC)} stretching backbone modes, assigned to proline and hydroxyproline in collagen.

In one embodiment, data can be collected using invasive techniques wherein biopsy samples are collected. A typical punch biopsy is approximately 140 mm³. In another embodiment, Raman band data can be collected via non-invasive means, such as via a fiber optic probe-coupled system. In this embodiment, tissue volume of approximately 60 mm³, or greater, can be evaluated by Raman spectroscopy.

Obviously, many modifications and variations of the present invention are possible in light of the above teachings. It is therefore to be understood that, within the scope of the appended claims, the invention may be practiced otherwise than as specifically described.

What is claimed is:

1. A method for analyzing heterotopic ossification comprising determining the presence, maturity or composition of heterotopic ossification by determining one or more Raman spectral measurements of one or more Raman spectral bands, wherein said Raman spectral measurements are selected from the group consisting of band area, band center, band area ratios, band height, ratio of band height, increase or decrease from band center, and band area ratio.

2. The method of claim 1, wherein the one or more Raman spectral bands are selected from the group consisting of 1445 cm⁻¹, 945 cm⁻¹; 960 cm⁻¹; and 1070 cm⁻¹; and wherein two or more of said band areas are used in computing band area ratios, wherein said band area ratios are selected from the group consisting of 945 cm⁻¹/960 cm⁻¹, 1070 cm⁻¹/960 cm⁻¹, 960/1445, and 1070/1445 cm⁻¹.

3. The method of claim 1, wherein said determining of band area or computing of said band area ratios are made over a time period to determine the progression of heterotopic ossification.

4. The method of claim 1, wherein said determining of composition and maturity of heterotopic ossification is the detection of mineralization of soft tissue.

5. The method of claim 1, wherein said composition is mineral crystallinity, wherein said mineral crystallinity is determined by measuring the full width at half maximum of the 960 cm⁻¹ Raman spectral band.

6. The method of claim 1, wherein said method also includes the step of determining traumatized region of tissue prior to determining said area under multiple Raman vibrational bands associated with heterotopic ossification.

7. The method of claim 1, wherein said Raman spectral measurements are collected by invasive or noninvasive means.

8. The method of claim 1, wherein said Raman spectral bands comprise one or more bands selected from the group consisting of 1660, 1445, 1640, 1240, 1270, and 1340, and wherein said computing of band area ratios comprises one or more band area ratios selected from the group consisting of 1660/1445 cm⁻¹, 1680/1445 cm⁻¹, 1640/1445 cm⁻¹, 1640/1660 cm⁻¹, 1240/1270 cm⁻¹, and 1340/1270 cm⁻¹.

9. The method of claim 2, wherein said Raman spectral bands also comprises one or more of the vibrational bands selected from the group consisting of 591 cm⁻¹, 1003 cm⁻¹, 921 cm⁻¹, 876 cm⁻¹ 855 cm⁻¹ and 1045 cm⁻¹.

10. The method of claim 2, wherein mature heterotopic ossification is detected as a 50% decrease cm⁻¹ of the mineral-to-matrix ratio by measuring the band area ratio 960/1445 cm⁻¹.

11. The method of claim 2, wherein said early or mature HO is determined by an increase in said Raman band area ratio 1070/960 cm⁻¹ over said time period over normal bone.

12. The method of claim 2, wherein HO is determined as a greater than 3 cm⁻¹ increase of the 1445 cm⁻¹ band center.

13. The method of claim 2, wherein mature HO is detected as a 40% or greater decrease in 945/960 cm⁻¹ band area ratio from immature HO and wherein early HO is detected as at least a 40% increase in the 945/960 cm⁻¹ band area ratio over normal bone.

14. The method of claim 2, wherein mature HO is determined as a greater than 3 cm⁻¹ increase from the 1070 cm⁻¹ band center for early HO.

15. The method of claim 2, wherein mature HO is determined as a greater than 60% increase in the 1070/1445 cm⁻¹ band area ratio from normal bone.

16. The method of claim 2, wherein mature HO is determined as a greater than 2 cm⁻¹ decrease in the 945 cm⁻¹ band center normal bone.

17. The method of claim 2, wherein mature HO is determined by a greater than 5% increase compared to bone in mineral crystallinity, wherein mineral crystallinity is determined by 960 cm⁻¹ band width at half maximum.

18. The method of claim 2, wherein mature HO is determined by a 50% or greater increase in standard deviation compared to bone at the band area ration 960/1445 cm⁻¹.

19. The method of claim 6, wherein said determination of traumatized region of tissue is conducted by x-ray analysis or Raman spectral analysis.

20. The method of claim 7, wherein said data from invasive means includes the additional step of collection of one or more biopsy samples.

21. The method of claim 7, wherein said noninvasive means comprises a fiber optic probe-coupled system.

22. The method of claim 8, wherein mature HO is determined as a least a 60% decrease of the 1340/1270 cm⁻¹ band height ratio and wherein early HO is determined as at least a 20% decrease of the 1340/1270 cm⁻¹ band height compared to muscle.

23. The method of claim 8, wherein mature HO is determined as a 50% or greater decrease in the 1340/1270 cm⁻¹ band height ratio or the 1340/1270 cm⁻¹ band area ratio compared to early HO.

24. The method of claim 8, wherein mature HO is determined by a 25% or greater decrease in the 1660/1445 cm⁻¹ band area ratio compared to muscle or early HO.

25. The method of claim 8, wherein HO is determined by a two times or greater increase of the 1640/1660 cm⁻¹ band area ratio over muscle.

26. The method of claim 8, wherein HO is determined by a 2 cm⁻¹ increase from the 1660 cm⁻¹ band center of muscle.

27. The method of claim 9, wherein mature HO is determined as a greater than 10 cm⁻¹ increase from said 1045 cm⁻¹ band center.
